SEQUENCE LISTING (16 sequences)

SEQ ID NO: 1 - DNA, 1224 bp, Oceanospirillum kriegii
  CDS (1)..(1224)
atgggctattcagaccacttttttaaagctttgaatatatcggttgaagacaggaaaagtttgtctgagttttccaaaagaagcggtatacctgttaagaaattgaagtactacaacgaaggaaatgtagtacctaccggcaaggacctggaaaagatactctctaccgcgaacctttctgaggttttgcttcgcttgaaaatgggtaggctggataaggatattctagcggcaatacaggaaaatgcggaaagtgttcttgctgaaatcgacggttttaatccggttgtggattctccggaggtcgactgtacattggagtttgaaaccagactcggtaaactttatcgtggggattgttattctctactgaagtcaatggaaagcgattctgttgatctgatattctctgatcccccttttaatcttgacaagatatatccttctgatatggatgacaatataaaggtggataagtatattggctggagtcaggagtggataaaggaatgcgctcgtgttttaaagcctggtggtgcgcttttcatgtggaacctcccgaagtggaatgtggcattaggttcgtttgttgatggcctgcttacgttcagaaactggattggcgtagacataaaatatagccttccaattagaaatcgattgtatccatctcattattcgttgatgtattacatcaagggtgaaaagccgaattcctttcatccagaccgtttggctatggatgtttgcccaaagtgctacggcgatttgaaagattatggcggttacaaggataagatgaatccgttgggtattaatctttctgatgtctggtatgacattcctcctgtaaggcatgcaaagtacaaaaggagaaagggctccaatgagctttcgttaaagctgttggacaggatcattgagatggcttcagacgaaggtgatttggtgtttgatccattcgggggctccggcacaacgtatatggcagccgagctaaagggccggagatgggttggctgtgaactgggaccaacagatattattaaagagcgattttctttgatcgaagaagaaagggatatactcaatggttatcgagggcgagtaaatgctcttttccctgagaaaaccagatccgagcgagaaaaacgtggtttgtggacttgtgagacttttagcaaaaacgaacagtcggaactctttgacaaaaacctgaagtaa

SEQ ID NO: 2 - protein, 407 aa, Oceanospirillum kriegii
MGYSDHFFKALNISVEDRKSLSEFSKRSGIPVKKLKYYNEGNVVPTGKDLEKILSTANLSEVLLRLKMGRLDKDILAAIQENAESVLAEIDGFNPVVDSPEVDCTLEFETRLGKLYRGDCYSLLKSMESDSVDLIFSDPPFNLDKIYPSDMDDNIKVDKYIGWSQEWIKECARVLKPGGALFMWNLPKWNVALGSFVDGLLTFRNWIGVDIKYSLPIRNRLYPSHYSLMYYIKGEKPNSFHPDRLAMDVCPKCYGDLKDYGGYKDKMNPLGINLSDVWYDIPPVRHAKYKRRKGSNELSLKLLDRIIEMASDEGDLVFDPFGGSGTTYMAAELKGRRWVGCELGPTDIIKERFSLIEEERDILNGYRGRVNALFPEKTRSEREKRGLWTCETFSKNEQSELFDKNLK

SEQ ID NO: 3 - DNA, 585 bp, Oceanospirillum kriegii
  CDS (1)..(585)
gtgaaaataaagcgtattgaggtccttataaataatggatcggttccagggattcctatgatcttgaatgaaattcaagatgcgataaaaacagtttcttggccagaaggtaataattcattcgttattaatcctgttcgcaaaggtaatggtgttaaaccaattaaaaattcctgtatgagacatcttcatcagaaaggctgggctcttgaacatcctgttagaattaaggctgaaatgaggccgggcccattggatgcggtgaagatgattggagggaaagcattcgcacttgagtgggagacggggaatatatcatcgtcgcatagggcaattaataaaatggtcatggggatgttggaacgtgtgattatcggaggtgttttgattcttccatcaagggatatgtacaactacttgactgatagggtaggtaattttagagagctggaaccttatttctcagtttggcggcagtttaatttgaaagatgcttatcttgctattgttgaaattgaacatgatagtgtcgatgcgcaggtttcattaattcctaagggtacagatggtcgtgctataaggtga

SEQ ID NO: 4 - protein, 194 aa, Oceanospirillum kriegii
VKIKRIEVLINNGSVPGIPMILNEIQDAIKTVSWPEGNNSFVINPVRKGNGVKPIKNSCMRHLHQKGWALEHPVRIKAEMRPGPLDAVKMIGGKAFALEWETGNISSSHRAINKMVMGMLERVIIGGVLILPSRDMYNYLTDRVGNFRELEPYFSVWRQFNLKDAYLAIVEIEHDSVDAQVSLIPKGTDGRAIR

SEQ ID NO: 5 - DNA, 237 bp, Oceanospirillum kriegii
  CDS (1)..(237)
atgaaagtggaatgtgcatttggaagaatcctaaagcagcttagaacagcaaaagggctatcgcaggagcaactagccctgagctgtggcttggaccgtacatttatttcaatgttagaaagagggcaaaggcagccgtctttatcgtctatcctctccctatcaaaatcgcttgaaacacctgcgcacgagatgctaaagaaaactacggatttaatagactcagaaaaatcttaa

SEQ ID NO: 6 - protein, 78 aa, Oceanospirillum kriegii
MKVECAFGRILKQLRTAKGLSQEQLALSCGLDRTFISMLERGQRQPSLSSILSLSKSLETPAHEMLKKTTDLIDSEKS

SEQ ID NO: 7 - DNA, 24 bp, Synthetic
tgcattaacaggacttcaatcacc

SEQ ID NO: 8 - DNA, 24 bp, synthetic
acataatgcaggccacgcccaacc

SEQ ID NO: 9 - DNA, 24 bp, synthetic
cggtctggatgaaaggaattcggc

SEQ ID NO: 10 - DNA, 24 bp, synthetic
aatctttcaaatcgccgtagcact

SEQ ID NO: 11 - DNA, 24 bp, synthetic
tcctgtatgagacatcttcatcag

SEQ ID NO: 12 - DNA, 24 bp, synthetic
caccattacctttgcgaacaggat

SEQ ID NO: 13 - DNA, 42 bp, synthetic
ggaggagtccatatgaaaataaagcgtattgaggtccttata

SEQ ID NO: 14 - DNA, 42 bp, synthetic
ggaggagtcgactcaccttatagcacgaccatctgtaccctt

SEQ ID NO: 15 - DNA, 42 bp, syntheti; identical to SEQ ID NO: 13

SEQ ID NO: 16 - DNA, 27 bp, synthetic
ccttatagcacgaccatctgtaccctt

(19) United States
(12) Patent Application Publication
(10) Pub. No.: US 2004/0175816 A1
(43) Pub. Date: Sep. 9, 2004

(54) Method for cloning and expression of OkrAI restriction endonuclease and methyltransferase

(75) Inventors: Shuang-Yong Xu, Lexington, MA (US); Jian-Ping Xiao, Wenham, MA (US); Rebecca B. Kucera, Hamilton, MA (US); James Samuelson, Danvers, MA (US)

Correspondence Address: Gregory D. Williams, General Counsel, New England Biolabs, Inc., 32 Tozer Road, Beverly, MA, 01915, US

(73) Assignee: New England Biolabs, Inc.

(21) Appl. No.: 10/378,124

(22) Filed: Mar. 3, 2003

(51) Int. Cl. C12N 9/22; C07H 21/04; C12N 1/21; C12N 15/74

(52) U.S. Cl. 435/199; 435/69.1; 435/252.3; 435/320.1; 536/23.2

(57) ABSTRACT

The present invention relates to recombinant DNA encoding the OkrAI restriction endonuclease as well as OkrAI methylase, expression of OkrAI restriction endonuclease in E. coli cells containing the recombinant DNA.

### BACKGROUND OF THE INVENTION

The present invention relates to recombinant DNA that encodes the OkrAI restriction endonuclease (OkrAI endonuclease or OkrAI) as well as OkrAI methyltransferase (OkrAI methylase or M. OkrAI), expression of OkrAI endonuclease and methylase in E. coli cells containing the recombinant DNA.

OkrAI endonuclease is found in the strain of Oceanospirillum kriegii (ATCC 35192). It recognizes the double-stranded DNA sequence 5′G/GATCC3′ (/ indicates the cleavage position) and cleaves between two Gs to generate a 4-base 5′ overhang. OkrAI methylase is also found in the same strain, which recognizes the same DNA sequence and presumably modifies the cytosine at the N4 position on hemi-methylated or non-methylated GGATCC sites.

Type II restriction endonucleases are a class of enzymes that occur naturally in bacteria and in some viruses. When they are purified away from other bacterial/viral proteins, restriction endonucleases can be used in the laboratory to cleave DNA molecules into small fragments for molecular cloning and gene characterization.

Restriction endonucleases recognize and bind particular sequences of nucleotides (the ‘recognition sequence’) along the DNA molecules. Once bound, they cleave the molecule within (e.g. BamHI), to one side of (e.g. SapI), or to both sides (e.g. TspRI) of the recognition sequence. Different restriction endonucleases have affinity for different recognition sequences. Over two hundred and twenty-eight restriction endonucleases with unique specificities have been identified among the many hundreds of bacterial species that have been examined to date (Roberts and Macelis, Nucl. Acids Res.29:268-269 (2001)).

Restriction endonucleases typically are named according to the bacteria from which they are discovered. Thus, the species Deinococcus radiophilus for example, produces three different restriction endonucleases, named DraI, DraII and DraIII. These enzymes recognize and cleave the sequences 5′TTT/AAA3′, 5′PuG/GNCCPy3′ and 5′ CACNNN/GTG3′ respectively. Escherichia coli RY13, on the other hand, produces only one enzyme, EcoRI, which recognizes the sequence 5′G/AATTC3′.

A second component of bacterial/viral restriction-modification (R-M) systems is the methylase. These enzymes co-exist with restriction endonucleases and they provide the means by which bacteria are able to protect their own DNA and distinguish it from foreign DNA. Modification methylases recognize and bind to the same recognition sequence as the corresponding restriction endonuclease, but instead of cleaving the DNA, they chemically modify one particular nucleotide within the sequence by the addition of a methyl group (C5 methyl cytosine, N4 methyl cytosine, or N6 methyl adenine). Following methylation, the recognition sequence is no longer cleaved by the cognate restriction endonuclease. The DNA of a bacterial cell is always fully modified by the activity of its modification methylase. It is therefore completely insensitive to the presence of the endogenous restriction endonuclease. Only unmodified, and therefore identifiably foreign DNA, is sensitive to restriction endonuclease recognition and cleavage. During and after DNA replication, usually the hemi-methylated DNA (DNA methylated on one strand) is also resistant to the cognate restriction digestion.

With the advancement of recombinant DNA technology, it is now possible to clone genes and overproduce the enzymes in large quantities. The key to isolating clones of restriction endonuclease genes is to develop an efficient method to identify such clones within genomic DNA libraries, i.e. populations of clones derived by ‘shotgun’ procedures, when they occur at frequencies as low as 10⁻³ to 10⁻⁴. Preferably, the method should be selective, such that the unwanted clones with non-methylase inserts are destroyed while the desirable rare clones survive.

A large number of type II restriction-modification systems have been cloned. The first cloning method used bacteriophage infection as a means of identifying or selecting restriction endonuclease clones (EcoRII: Kosykh et al., Mol. Gen. Genet. 178:717-719 (1980); HhaII: Mann et al., Gene 3-:97-112 (1978); PstI: Walder et al., Proc. Sat. Acad. Sci. 78:1503-1507 (1981)). Since the expression of restriction-modification systems in bacteria enables them to resist infection by bacteriophages, cells that carry cloned restriction-modification genes can, in principle, be selectively isolated as survivors from genomic DNA libraries that have been exposed to phage. However, this method has been found to have only a limited success rate. Specifically, it has been found that cloned restriction-modification genes do not always confer sufficient phage resistance to achieve selective survival.

Another cloning approach involves transferring systems initially characterized as plasmid-borne into E. coli cloning vectors (EcoRV: Bougueleret et al., Nucl. Acids. Res. 12:3659-3676 (1984); PaeR7: Gingeras and Brooks, Proc. Natl. Acad. Sci. USA 80:402-406 (1983); Theriault and Roy, Gene 19:355-359 (1982); PvuII: Blumenthal et al., J. Bacteriol. 164:501-509 (1985); Bsa45I: Wayne et al. Gene 202:83-88 (1997)).

A third approach is to select for active expression of methylase genes (methylase selection) (U.S. Pat. No. 5,200,333 and BsuRI: Kiss et al., Nucl. Acids. Res. 13:6403-6421 (1985)). Since restriction-modification genes are often closely linked together, both genes can often be cloned simultaneously. This selection does not always yield a complete restriction system however, but instead yields only the methylase gene (BspRI: Szomolanyi et al., Gene 10:219-225, (1980); BcnI: Janulaitis et al., Gene 20:197-204 (1982); BsuRI: Kiss and Baldauf, Gene 21:111-119, (1983); and BsaI: Walder et al., J. Biol. Chem. 258:1235-1241 (1983)).

A more recent method, the “endo-blue method”, has been described for direct cloning of thermostable restriction endonuclease genes into E. coli based on the indicator strain of E. coli containing the dinD::lacZ fusion (Fomenkov et al., U.S. Pat. No. 5,498,535; Fomenkov et al., Nucl. Acids Res. 22:2399-2403 (1994)). This method utilizes the E. coli SOS response signals following DNA damage caused by restriction endonucleases or non-specific nucleases. A number of thermostable nuclease genes (TaqI, Tth111I, BsoBI, Tf nuclease) have been cloned by this method (U.S. Pat. No. 5,498,535). The disadvantage of this method is that some positive blue clones containing a restriction endonuclease gene are difficult to culture due to the lack of the cognate methylase gene.

There are three major groups of DNA methyltransferases based on the position and the base that is modified (C5 cytosine methylases, N4 cytosine methylases, and N6 adenine methylases). N4 cytosine and N6 adenine methylases are amino-methyltransferases (Malone, et al. J. Mol. Biol. 253:618-632, (1995)). When a restriction site on DNA is modified (methylated) by the methylase, it is resistant to digestion by the cognate restriction endonuclease. Sometimes methylation by a non-cognate methylase can also confer DNA sites resistant to restriction digestion. For example, Dcm methylase modification of 5′ CCWGG3′ (W=A or T) can also make the DNA resistant to PspGI restriction digestion. Another example is that CpM methylase can modify the CG dinucleotide and make the NotI site (5′GCGGCCGC3′) refractory to NotI digestion (New England Biolabs' catalog, 2000-01, page 220). Therefore methylases can be used as a tool to modify certain DNA sequences and make them uncleavable by restriction enzymes.

Type II methylase genes have been found in many sequenced bacterial genomes (GenBank, http://www.ncbi.nlm.nih.gov; and REBASE®, http://rebase.neb.com/rebase). Direct cloning and over-expression of ORFs adjacent to the methylase genes yielded restriction enzymes with novel specificities (Kong et al. Nucl. Acids Res. 28:3216-3223 (2000)). Thus microbial genome mining emerged as a new way of screening/cloning new type II restriction enzymes and methylases and their isoschizomers.

Because purified restriction endonucleases and modification methylases are useful tools for creating recombinant molecules in the laboratory, there is a strong commercial interest to obtain bacterial strains through recombinant DNA techniques that produce large quantities of restriction enzymes and methylases. Such over-expression strains should also simplify the task of enzyme purification.

### SUMMARY OF THE INVENTION

The present invention relates to a method for cloning OkrAI restriction endonuclease gene (okrAIR which is an isoschizomer of BamHI) from Oceanospirillum kriegii into E. coli by methylase selection, inverse PCR, and direct PCR from genomic DNA using primers based on the DNA sequences obtained via methylase selection.

Initial attempts to clone OkrAI by methylase selection were unsuccessful. Specifically, two BamHI resistant clones were first identified in an EcoRI genomic DNA library. However, they turned out to be false positive since they had suffered DNA rearrangements and the DNA inserts did not encode any DNA methylase or endonuclease.

The okrAIM gene was successfully cloned and selected from an ApoI genomic DNA library by the methylase selection method. Specifically, the entire insert was digested with restriction enzyme ApoI and the resulting fragments were subcloned and sequenced. Deletion clones were also constructed to facilitate the sequencing efforts. The okrAIM gene encoding an N4 cytosine (N4C) methylase was identified within the insert. A small ORF encoding a putative control protein (okrAIC gene) was also found. Further upstream a partial ORF was found that has amino acid sequence similarity to BamHI endonuclease. Since OkrAI is an isoschizomer of BamHI, it was reasoned that they may share some amino acid sequence similarity. To obtain the entire sequence inverse PCR primers were synthesized based on the truncated gene sequence. Inverse PCR product carrying the remaining coding sequence was obtained in the AluI digested and self-ligated template. After gel purification, the PCR product was sequenced and the okrAIR gene was cloned into a T7 expression vector pAII17S. OkrAI endonuclease was over-expressed and the over-produced protein (22 kDa) was detected in SDS-PAGE. OkrAI endonuclease was also fused to an intein and a chitin binding domain so that OkrAI can be purified via affinity tag and chitin column chromatography.

### BRIEF DESCRIPTION OF THE DRAWINGS

FIG. 1. Gene organization of OkrAI restriction-modification system. okrAIR, OkrAI restriction endonuclease gene; okrAIM, OkrAI methylase gene.

FIG. 2. OkrAI methylase gene sequence (okrAIM, 1224 bp) (SEQ ID NO:1) and its encoded amino acid sequence (SEQ ID NO:2).

FIG. 3. OkrAI endonuclease gene sequence (okrAIR, 585 bp) (SEQ ID NO:3) and its encoded amino acid sequence (SEQ ID NO:4).

FIG. 4. OkrAI C gene sequence (okrAIC, 237 bp) (SEQ ID NO:5) and its encoded amino acid sequence (SEQ ID NO:6).

FIG. 5. Purified recombinant OkrAI endonuclease analyzed on SDS-PAGE. Lane 1, protein size marker; Lanes 3 to 6, 40, 20, 5, and 1 μg OkrAI endonuclease protein, respectively. The apparent molecular mass of OkrAI is 22 kDa.

FIG. 6. OkrAI endonuclease activity assay on λ DNA in the presence of 1 to 10% glycerol. Lane 1, λ DNA; lanes 2 to 11, 1% to 10% of glycerol; lane 12, DNA size marker. Assay condition: 100 units of OkrAI to digest 1 μg λ DNA in 1× NEB buffer 3 at room temperature (r.t.) for 1 h.

FIG. 7. OkrAI activity assay following heat-treatment. Lane 1, λ DNA; lane 2, 1 μg λ DNA digested with 20 units of OkrAI; lane 3, 1 μ g λ DNA treated with heat-treated OkrAI (20 units); lane 4, 1 μg λ DNA digested with 100 units of okrAI; lane 5, 1 μg λ DNA treated with heat-treated OkrAI (100 units). Assay condition: OkrAI or heat-treated OkrAI to digest 1 μg λ DNA in 1× NEB buffer 3 at r.t. for 1 h in a total volume of 50 μl. 20 and 100 units of OkrAI were first heated at 65° C. for 30 min.

### DETAILED DESCRIPTION OF THE INVENTION

1. Preparation of Genomic DNA, Restriction Digestion, and Construction of Genomic DNA Library

Genomic DNA was prepared from Oceanospirillum kriegii by the standard method. ApoI endonuclease was used to partially digest the genomic DNA. ApoI fragments in 2-10 kb were purified and ligated to EcoRI digested and CIP treated pRRS vector that contains multiple OkrAI sites. The ligated DNA was used to transform ER2502 by electroporation. Approximately 10,000 Ap^{R} transformants were derived from the transformation. All the colonies were pooled and amplified. Plasmid DNA was prepared to generate a plasmid DNA library.

2. Cloning of okrAIM Gene by Methylase Selection

The primary plasmid library was challenged with BamHI. The digested DNA was transferred into ER2502 by transformation, resulting in ˜100 Ap^{R} survivors. Plasmid DNA was prepared from cultures of the Ap^{R} survivors. Following BamHI digestion and agarose gel electrophoresis one resistant clone was detected.

3. Restriction Mapping and Subcloning of the Insert

The resistant plasmid DNA was digested with a number of restriction enzymes to estimate the insert size. The insert size was estimated to be ˜2.8 kb. Multiple ApoI fragments from the insert were gel-purified and subcloned into pUC19. The inserts were sequenced using pUC universal primers. HincII and HindIII fragment deletion clones were also constructed and sequenced. One ORF has high homology to the N4C methylase family and this ORF was named okrAIM gene. A second ORF has sequence homology to the family of R-M system control genes (okrAIC gene). One truncated ORF (˜400 bp) was also found. This partial ORF was a candidate for the okrAIR gene.

4. Inverse PCR Amplification of DNA Upstream of okrAIM and okrAIC Gene

After identification of the methylase gene, efforts were made to clone adjacent upstream DNA. Inverse PCR primers were made based on the known truncated sequence.

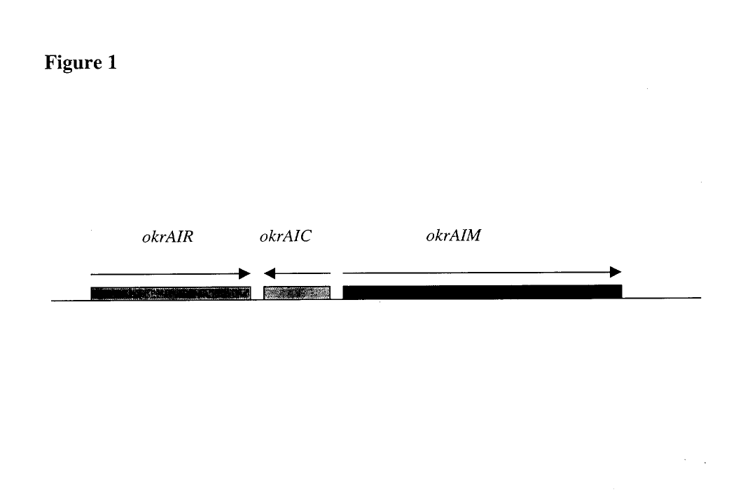

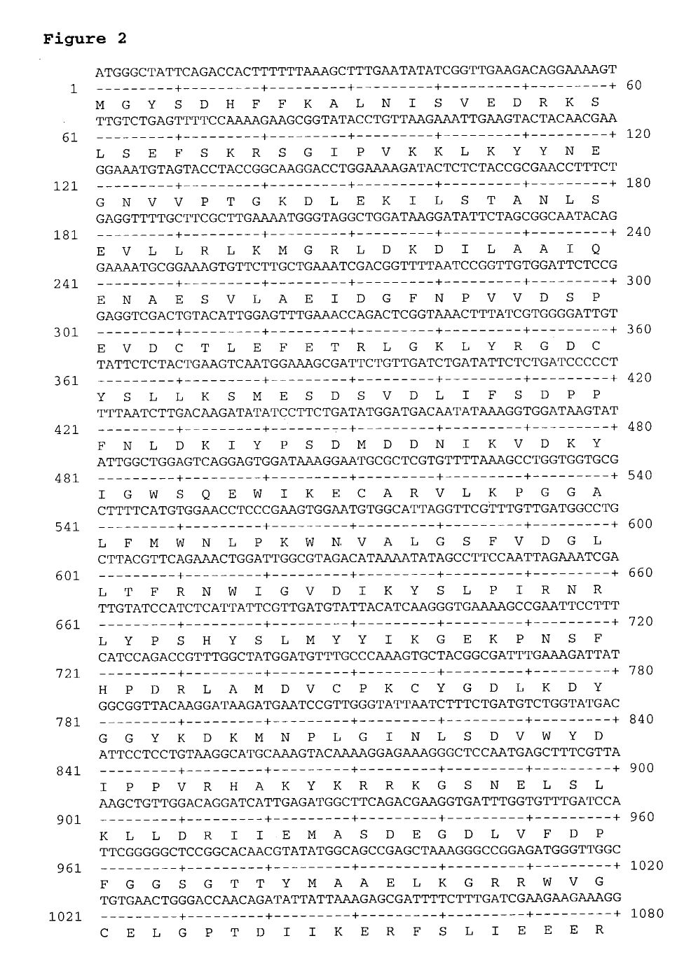

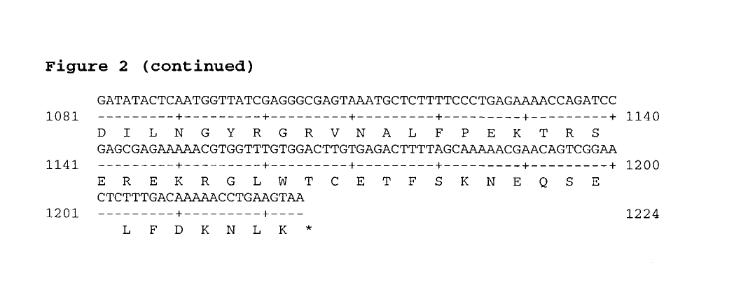

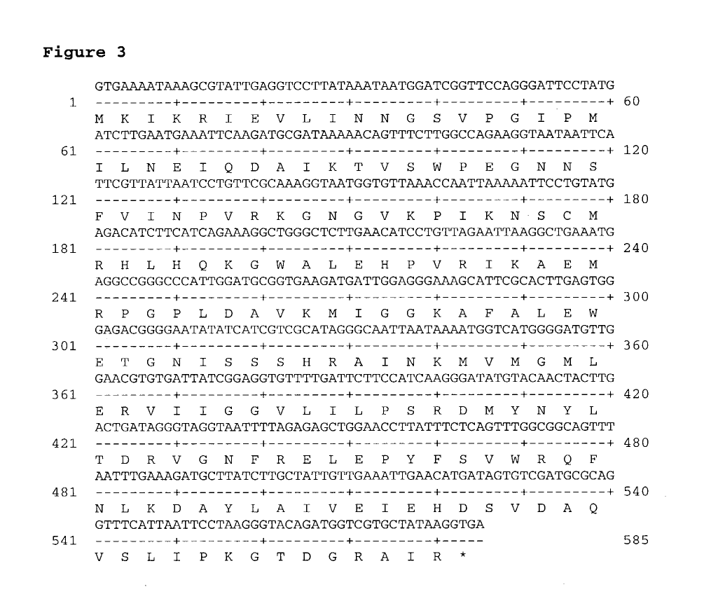

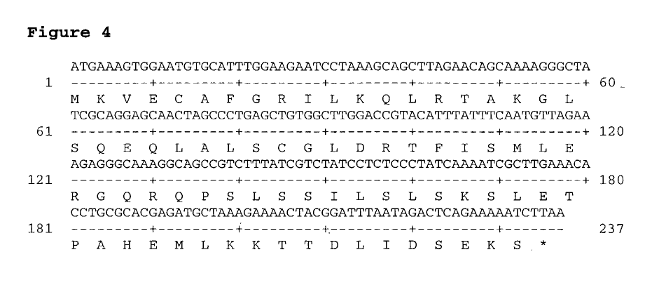

The genomic DNA was digested with 4-base cutting restriction enzymes, purified, and self-ligated. The circular DNA molecules were used as templates for inverse PCR. PCR product was found using the AluI digested and ligated template. The PCR product was purified and sequenced directly with the primers for the inverse PCR reaction. The AluI fragment generated about 400 bp new DNA sequence. A 585-bp ORF was found and named the okrAIR gene. Transcription of M and R genes is oriented in the same direction (see FIG. 1 for gene organization).

5. Expression of okrAIR Gene in E. coli

The non-cognate methylase, M. BamHI, was used to pre-modify E. coli host ER2566 for OkrAI endonuclease expression. The okrAIR gene was cloned in a T7 expression vector pAII17S. The okrAIR gene was amplified by PCR, digested with NdeI and SalI and ligated to pAII17 with compatible ends, and transformed into the M. BamHI modified host ER2566. Alternatively, the okrAIR gene was amplified in PCR and digested with NdeI and ligated to NdeI and SmaI digested pTYB2 expression vector. In pTYB2 the okrAIR gene is fused to an intein from Saccharomyces cerevisiae VMA1 gene and chitin binding domain. Therefore OkrAI endonuclease can be purified by chitin column chromatography. After the fusion protein is bound to the chitin column, the OkrAI endonuclease can be cleaved from the fusion by addition of DTT. The endonuclease gene insert in pAII17S or pTYB2 was sequenced and confirmed to be the wild-type coding sequence. The final yield from the T7 expression system, ER2566 [pACYC-BamHIM, pAII17S-OkrAIR], is approximately 5×10⁶ units per gram of wet IPTG-induced cells.

The present invention is further illustrated by the following Examples. These Examples are provided to aid in the understanding of the invention and are not construed as a limitation thereof.

The references cited above and below are herein incorporated by reference.

### EXAMPLE 1

### Cloning of OkrAI Restriction-Modification System in E. coli

1. Preparation of Genomic DNA

Genomic DNA was prepared from Oceanospirillum kriegii (ATCC 35192) by the standard procedure consisting of the following steps:

a. Cell lysis by addition of lysozyme (2 mg/ml final), sucrose (1% final), and 50 mM Tris-HCl, pH 8.0.

b. Further cell lysis by addition of SDS at a final concentration of 0.1%.

c. Further cell lysis by addition of 1% Triton X-100, 62 mM EDTA, 50 mM Tris-HCl, pH 8.0.

d. Removal of proteins by phenol-CHCl₃ extraction of DNA 3 times (equal volume) and CHCl₃ extraction once.

e. Dialysis in 4 liters of TE buffer, buffer change twice.

f. RNase A treatment to remove RNA.

g. Genomic DNA precipitation in 95% ethanol, centrifuged, washed, dried and resuspended in TE buffer.

2. Restriction Digestion of Genomic DNA and Construction of Genomic DNA Library

EcoRI Complete Library:

EcoRI digested genomic DNA was ligated to EcoRI cut and CIP treated pEZL38 vector DNA (pEZL38 carries two OkrAI sites) at 16° C. overnight. The ligated DNA was transferred into ER2267 by transformation. Transformants were plated on Ap plates overnight at 37° C. Approximately 22,00 Ap^{R} colonies were pooled. Plasmids were purified from the cells by alkaline lysis method. Five ug of plasmid were digested with 3 units or 30 unitps of BamHI at 37° C. overnight. The challenged DNA was used to transform ER2677. Ap^{R} survivor transformants were amplified in small cultures and plasmids were purified from individual cultures and digested with Ba HI to test resistance to BamHI digestion. Two resistant clones (#18 and #21) were identified and later proved to be false positive. These two clones do not display M. OkrAI and OkrAI endonuclease activities.

ApoI Partial Library:

Varying units of restriction enzymes ApoI were used to digest 5 μg genomic DNA (ApoI: 2, 1, 0.5, 0.25, 0.125 units) at 50° C. for 1 h to achieve limited partial digestion. It was found that 0.5 and 0.25 units of ApoI gave rise to optimal partial digestion. The partially digested DNA in the range of 2 kb to 10 kb was gel-purified from a low-melting agarose gel. Following β-agarase treatment and ethanol precipitation, the ApoI digested DNA was ligated to EcoRI and CIP treated pRRS vector that contains multiple OkrAI sites. The vector pRRS is a pUC-based high-copy-number plasmid for cloning and expression of genes in E. coli. The ligated DNA was used to transform EndA⁻ RR1 competent cells (ER2502, New England Biolabs, Inc. collection (Beverly, Mass.)) by electroporation. Approximately 10,000 Ap^{R} transformants were obtained for the partial library. All the colonies were pooled and amplified in 1 liter LB+Ap (100 μg/ml) overnight. Plasmid DNA was prepared by Qiagen (Studio City, Calif.) Maxi-prep columns, generating a plasmid DNA library.

3. Cloning of OkrAIM Gene by Methylase Selection

The primary plasmid DNA library (1 μg, 2 μg, and 5 μg DNA) was challenged with 100 units of BamHI at 37° C. overnight in a total volume of 100 l in 1× BamHI buffer (150 mM NaCl, 50 mM Tris-HCl, 10 mM MgCl₂, 1 mM DTT). Because BamHI and OkrAI are isoschizomer, it was predicted that M. OkrAI modified plasmid DNA might be resistant to BamHI digestion. The BamHI digested DNA was transferred into ER2502 by transformation, generating ˜100 Ap^{R} survivors. Plasmid DNA was prepared by Qiagen (Studio City, Calif.) spin columns from 36 overnight cell cultures (1.5 ml×36). After digestion of the plasmid DNA by BamHI, one complete resistant clone (#30) was detected and it was further characterized.

4. Restriction Mapping and Subcloning of the Insert

The resistant plasmid DNA was digested with restriction enzymes ApoI, BamHI, EcoRI, HindIII, PvuII, PstI, SacI, and SphI to estimate the insert size. The insert size was estimated to be ˜2.8 kb. The BamHI-resistant plasmid was digested with ApoI and four ApoI fragments were gel-purified from a low-melting agarose gel and sub-cloned into EcoRI digested and CIP treated pUC19. The ApoI fragment inserts were screened and sequenced using pUC forward and reverse universal primers.

Two deletion clones were also constructed. HincII fragment and HindIII fragment deletion clones were also constructed by HincII or HindIII digestion of the resistant clone and self-ligation at a low DNA concentration. After retransformation and confirmation of each deletion, the deletion clones were also sequenced by pUC primers and custom made primers. DNA sequencing was performed using the dye terminator sequencing kit from PE Biosystems (Foster City, Calif.). The sequencing primers have the following sequences:

| | |
|---|---|
| 5′TGCATTAACAGGACTTCAATCACC3′ | (SEQ ID NO: 7) |
| 5′ACATAATGCAGGCCACGCCCAACC3′ | (SEQ ID NO: 8) |
| 5′CGGTCTGGATGAAAGGAATTCGGC3′ | (SEQ ID NO: 9) |
| 5′AATCTTTCAAATCGCCGTAGCACT3′ | (SEQ ID NO: 10) |

One open reading frame (ORF) has amino acid sequence homology to the N4 cytosine methylase family and BamHI methylase. This ORF was named the okrAIM gene. A second ORF has sequence homology to the control gene (C gene) of some restriction-modification systems. One truncated ORF (˜400 bp) was also found. This partial ORF was a candidate for the okrAIR gene. Therefore, major efforts were made to clone the remaining part of the truncated ORF.

5. Inverse PCR Amplification of DNA Downstream of OkrAI Methylase

After identification of the methylase gene, it is desirable to clone the adjacent genes in order to find the cognate endonuclease gene. The genomic DNA was digested with 4-base cutting restriction enzymes AluI, HhaI, NlaIII, RsaI, Sau3AI, and TaqI, respectively in appropriate restriction buffers and temperatures. TaqI digestion was performed at 65° C. and the rest at 37° C.

The digested DNA was purified through Qiagen (Studio City, Calif.) spin columns. DNA self-ligation was performed at a low DNA concentration (2 μg/ml) overnight at 16° C. T4 DNA ligase was inactivated at 65° C. for 30 min and the circular DNA was precipitated in ethanol and used as the template for inverse PCR. Two primers were synthesized with the following sequences:

| | | |
|---|---|---|
| 5′TCCTGTATGAGACATCTTCATCA | (184-32) | (SEQ ID NO: 11) |
| G3′ | | |
| 5′CACCATTACCTTTGCGAACAGGA | (184-33) | (SEQ ID NO: 12) |
| T3′ | | |

PCR conditions were 95° C. for 1 min, 50° C. for 1 min, 72° C. for 2 min for 40 cycles. A PCR product (˜450-500 bp) was found in the AluI template. It was purified from a low-melting agarose gel, treated with β-agarase for 2 h, precipitated with ethanol, resuspended in TE buffer, and sequenced directly with primers 184-32 and 184-33. Sequencing of the AluI fragment generated ˜400 bp new DNA sequence. A start codon and a stop codon were found in the ORF and this ORF was named the okrAIR gene (gene size 585 bp). Transcription of R and M genes is oriented in the same direction (see FIG. 1 for gene organization). The orientation of the okrAIC gene is opposite of the okrAIR gene.

### EXAMPLE 2

### Expression of okrAIR Gene in E. coli Using T7 Expression System (pAII17S Vector)

Two PCR primers were synthesized for the amplification of the okrAIR gene. The primers have the following sequences:

| | | |
|---|---|---|
| 5′GGAGGAGTCCATATGAAAATAA | (190-121) | (SEQ ID NO: 13) |
| AGCGTATTGAGGTCCTTATA3′ | | |
| 5′GGAGGAGTCGACTCACCTTATA | (190-122) | (SEQ ID NO: 14) |
| GCACGACCATCTGTACCCTT 3′ | | |

An NdeI site and a SalI site were incorporated in the forward and reverse primers, respectively. The okrAIR gene was amplified from the genomic DNA in a PCR reaction under the conditions: 95° C. for 1 min, 60° C. for 1 min, 72° C. for 1 min for 20 cycles; 2 units of Vent® DNA polymerase with varying concentrations of Mg++ (2 to 10 mM) in a total volume of 100 μl. The PCR DNA was purified by phenol-chloroform extraction and chloroform extraction and ethanol precipitation. It was then digested with restriction enzymes NdeI and SalI for 3 h at 37° C. The DNA was purified further by phenol-chloroform extraction and chloroform extraction and ethanol precipitation. The PCR DNA was ligated to a modified T7 expression vector pAII17S. Vector pAII17S was derived from pAII17. The BamHI site in pAII17 was filled-in with Klenow fragment and a SalI linker was inserted to replace the BamHI site (Xu, S.-Y. New England Biolabs, Beverly, Mass.). The cloning sites in pAII17S are NdeI and SalI sites. Ligated PCR DNA/vector pAII17S were transferred into pre-modified expression host ER2566 [pACYC184-bamHIM] by transformation. Thirty-six transformants were screened, but none of them contained the desired okrAIR gene insert. To increase the cloning efficiency, the PCR DNA was digested again with NdeI and SalI overnight in 1× NEB buffer 4 (>16 h). The PCR DNA was purified and ligated to pAII17S with compatible ends. The ligated DNA was then transferred into E. coli host ER2566 [pACYC184-bamHIM]. This time after screening 18 transformants, 17 clones with inserts were found. Ten ml of cell cultures were grown for 10 isolates to late log phase and IPTG was added to a final concentration of 0.5 mM to induce OkrAI production. IPTG induction continued for 3 h and cells were collected by centrifugation. Cell pellet was resupended in a sonication buffer (50 mM Tris-HCl, pH 7.8, 10 mM β-mercaptoethanol, 50 mM NaCl) and cell lysis was completed by sonication. Cell debris was removed by centrifugation and clarified cell lysate was used to assay OkrAI endonuclease activity on λ DNA substrate. All ten isolates displayed OkrAI endonuclease activity in cell extracts. Isolate #9 was further characterized in a stability study in 1 L culture. One ml of cells were inoculated into 1 L of LB+Ap+Cm and cultured overnight in a shaker at 37° C. Twenty ml of the overnight cells were inoculated into a fresh 1 L LB+Ap+Cm and shaken at 37° C. for 4 h to late log phase. IPTG was added to final concentration of 0.5 mM for 3 h. Cells were harvested and lysed by sonication and cell extract was assayed again for OkrAI activity λ DNA substrate. High OkrAI activity was detected following a thousand-fold dilution of the cell extract. The OkrAI activity yield is approximately 5×10⁶ units per gram of wet cells.

The plasmid DNA pAII17S-okrAIR was prepared by Qiagen (Studio City, Calif.) tip-20 column and the entire insert was sequenced. It was found that the insert contained the okrAIR wild-type coding sequence.

The strain NEB#1164 ER2566 [pACYC184-bamHIM, pAII17S-okrAIR] has been deposited under the terms and conditions of the Budapest Treaty with the American Type Culture Collection on Feb. 12, 2003 and received ATCC Accession No. PTA-5002.

### EXAMPLE 3

### Expression of OkrAI with an Intein and Chitin Binding Protein Fusion in pTYB2

Two PCR primers were synthesized for the amplification of the okrAIR gene. The primers have the following sequences:

| | | |
|---|---|---|
| 5′GGAGGAGTCCATATGAAAATAA | (190-121) | (SEQ ID NO: 15) |
| AGCGTATTGAGGTCCTTATA3′ | | |
| 5′CCTTATAGCACGACCATCTGTA | (191-21r) | (SEQ ID NO: 16) |
| CCCTT3′ | | |

An NdeI site was incorporated in the forward primer. OkrAIR gene was amplified from the genomic DNA in a PCR reaction under the conditions: 95° C. for 1 min, 60° C. for 1 min, 72° C. for 1 min for 20 cycles; 2 units of Vent® DNA polymerase with varying concentration of Mg⁺⁺ (2 to 10 mM) in a total volume of 100 μl. The PCR DNA was purified by phenol-chloroform extraction and chloroform extraction and ethanol precipitation. It was then digested with restriction enzyme NdeI for 3 h at 37° C. The DNA was purified further by phenol-chloroform extraction and chloroform extraction and ethanol precipitation. The vector DNA pTYB2 was digested with NdeI and SmaI for 3 h at 37° C. and further purified by phenol-chloroform extraction and chloroform extraction and ethanol precipitation. The PCR DNA (one NdeI end and one blunt end) was ligated to NdeI and SmaI digested pTYB2 and the ligated DNA was transferred into expression host ER2566 [pACYC184-bamHIM] by transformation. After screening 72 plasmids isolated from cultures of the transformants, eight clones were found to contain the desired PCR insert. These 8 clones with inserts were grown in 10 ml LB+Ap (50 μg/ml) and Cm (33 μg/ml) to late log phase. IPTG was added to a final concentration of 0.5 mM to induce OkrAI-intein-CBD fusion protein production. IPTG induction continued for 3 h and cells were collected by centrifugation. Cell pellet was resupended in a sonication buffer (50 mM Tris-HCl, pH 7.8, 10 mM β-mercaptoethanol, 50 mM NaCl) and cell lysis was completed by sonication. Cell debris was removed by centrifugation and the clarified cell lysate was incubated with DTT and used to assay OkrAI endonuclease activity on λ DNA substrate. The addition of DTT facilitates the cleavage of OkrAI from the fusion protein. Four isolates (#11, #13, #14, and #26) displayed high OkrAI endonuclease activity in cell extracts. Plasmid DNA pTYB2-okrAIR was prepared by Qiagen (Studio City, Calif.) tip-20 column and the entire insert was sequenced. It was found that each insert contained the wild-type coding sequence, except for the inclusion of a Gly codon at the C-terminal fusion junction. The intein-CBD-OkrAI fusion expression strain is ER2566 [pACYC184-bamHIM, pTYB2-okrAIR].

### EXAMPLE 4

### Large Scale Purification of OkrAI Endonuclease

All activity determinations were done by incubating OkrAI fractions with lambda DNA for specified times at 37° C.

A wet cell mass of 115 grams was suspended at 4° C. in buffer A (100 mM NaCl, 20 mM KPO4, (pH 6.8), 1 mM DTT, 1 mM EDTA). All subsequent procedures were done at 4° C. The cells were sonicated and the debris removed by centrifugation at 20,000× g for 1 hour.

The supernatant was applied to a 5×15 cm phosphocellulose column (Whatman P11) equilibrated in buffer A. After washing to remove unbound material, the activity was eluted with a 2000 ml gradient from 0.1 to 2 M NaCl. OkrAI activity eluted at approximately 0.9 M NaCl.

The peak tubes were combined and loaded directly onto a 5×6 cm hydroxylapatite (Bio-Rad Laboratories, (Richmond, Calif.)) column. After washing with buffer A to remove unbound material, it was determined that the OkrAI activity was in the flow-through and wash for the column. These fractions were combined and dialyzed against buffer B (100 mM NaCl, 10 mM Tris-HCl (pH 7.8), 1 mM DTT, 1 mM EDTA).

The dialyzed material was loaded onto a 5×14 cm DEAE column (Pharmacia (Milwaukee, Wis.)). After washing the column with buffer B, it was determined that the OkrAI activity was again in the flow-through and wash for the column.

These fractions were combined, diluted with buffer to lower the NaCl concentration to 50 mM NaCl and change the pH to 7.3, and loaded directly onto a 5×19 cm heparin column (Pharmacia (Milwaukee, Wis.)). After washing with buffer C, (50 mM NaCl, 10 mM Tris-HCl (pH 7.3), 1 mM DTT, 1 mM EDTA), the activity was eluted with a 2000 ml gradient from 50 mM to 1 M NaCl. OkrAI activity eluted at approximately 0.7 M NaCl. These fractions were combined and dialyzed against buffer B.

The dialyzed material was loaded onto a 2.5×10 cm Q-Sepharose (Pharmacia (Milwaukee, Wis.)) column. After washing with buffer B, it was determined that the OkrAI activity was in the flow-through and wash for the column.

The fractions were combined and loaded directly onto a 2.5×9 cm Affi-gel Blue (Bio-Rad Laboratories (Richmond, Calif.)) column. After washing the column with buffer B, the activity was eluted with a 500 ml gradient from 50 mM to 2 M NaCl. The activity eluted at a salt concentration of approximately 0.9 M NaCl. These fractions were combined and dialyzed against buffer C (150 mM NaCl, 10 mM Tris-HCl (pH 7.3), 1 mM DTT, 0.1 mM EDTA).

The dialyzed material was loaded onto a 1×4.5 cm Heparin (Pharmacia (Milwaukee, Wis.)) column. After washing the column with buffer C, the activity was eluted with a 200 ml gradient from 0.15M to 0.8M NaCl. The activity eluted at a salt concentration of approximately 0.5M. The fractions containing activity were dialyzed against buffer D (100 mM NaCl, 20 mM KPO4 (pH 6.8), 1 mM DTT, 0.1 mM EDTA)

The dialyzed material was loaded onto a 1×4 cm phosphocellulose (Whatman P11) column. After washing the column with buffer D, the activity was eluted with a 200 ml gradient from 0.1 M NaCl to 1 M NaCl. The activity eluted at a salt concentration of approximately 0.5 M NaCl.

The fractions containing activity were collected and dialyzed against buffer E (75 mM NaCl, 10 mM Tris-HCl (pH 7.5), 0.1 mM EDTA). After some precipitation was noted, the NaCl concentration was increased to 200 mM, the tris concentration was changed to 20 mM Tris-HCl (pH 8) 1 mM DTT and 5% (v/v) glycerol were added. Some remaining precipitated material was removed by centrifugation and 0.2 micron filtration.

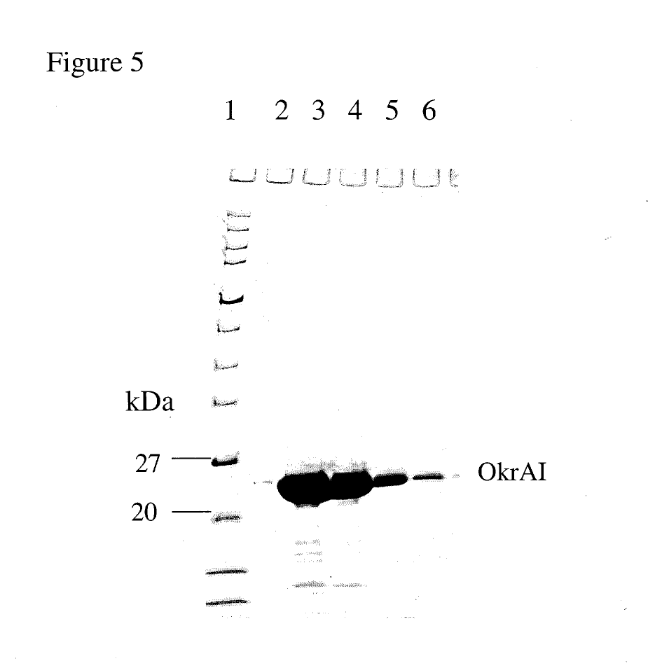

This final material was evaluated on SDS-PAGE (see FIG. 5) and by standard quality control assays and used for subsequent activity assays.

### EXAMPLE 5

### The Use of Room Temperature Incubation to Reduce OkrAI “Star” Activity

OkrAI displayed “star” activity at high glycerol concentration and prolonged incubation, a property similar to BamHI endonuclease. However, it was found that room temperature (r.t.) incubation dramatically reduced the OkrAI “star” activity. Room temperature (25° C.) reduced OkrAI specific activity only 2-fold, but the “star” activity was much reduced. In a 50 μl reaction volume, 20 units to 800 units OkrAI digestion of 1 μg of λ DNA did not show any “star” site cleavage at r.t. for 1 h.

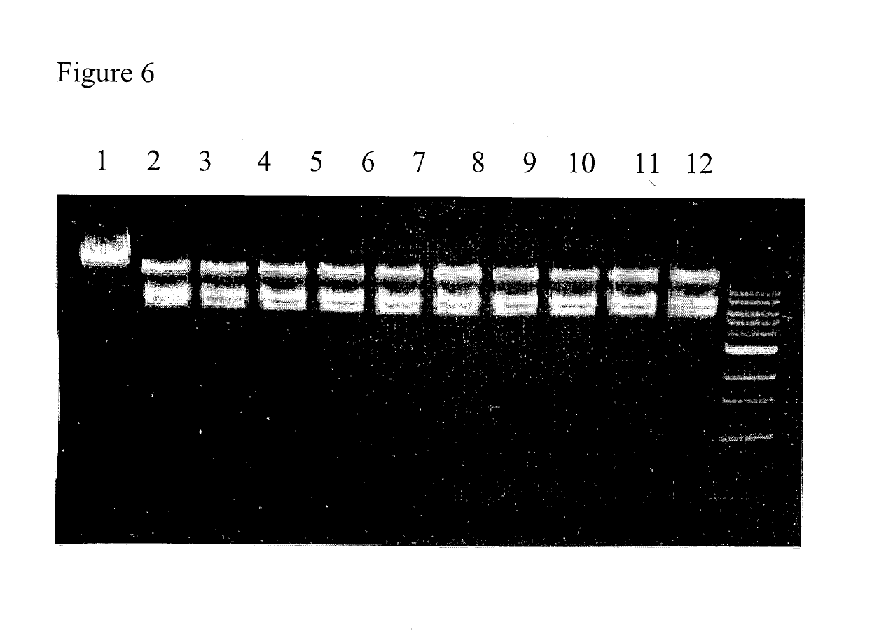

OkrAI “star” activity was also tested in the presence of 1-10% glycerol. The reaction conditions were: 100 units of OkrAI, 1× buffer 3 (100 mM NaCl, 50 mM Tris-HCl, 10 mM MgCl₂, 1 mM DTT), 1 μg of λ DNA, glycerol 1% to 10%, in a total volume of 50 μl at r.t. for 1 h. OkrAI does not display any “star” activity in the presence of 1% to 10% of glycerol at r.t. (see FIG. 6, lanes 2 to 11).

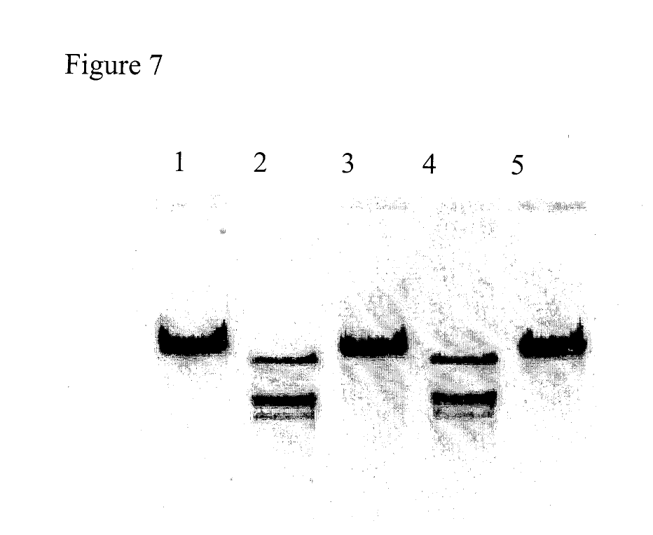

OkrAI endonuclease can be heat-inactivated at 65° C. for 30 min. 20 units and 100 units of OkrAI were first heated at 65° C for 30 min. Then they were incubated with 1 μg of λ DNA in 1× buffer 3 at r.t. for 1 h. After the heat treatment, no OkrAI endonuclease activity was detected, indicating that OkrAI was completely inactivated (see FIG. 7, lanes 3 and 5).

It was concluded that OkrAI displays no “star” activity at r.t. in the presence of 1-10% of glycerol (100 units of OkrAI tested). OkrAI does not show detectable “star” activity in over-digestion (20 to 800 units to digest 1 μg DNA). 100 units of OkrAI can be heat-inactivated at 65° C. for 30 min. The heat-inactivation property provides more convenience for molecular biology applications.

What is claimed is:

1. Isolated DNA encoding the OkrAI restriction endonuclease, wherein the isolated DNA is obtainable from Oceanospirillum kriegii.

2. A cloning vector comprising a vector into which a DNA segment encoding the OkrAI restriction endonuclease has been inserted.

3. Isolated DNA encoding the OkrAI restriction endonuclease, wherein the isolated DNA is obtainable from ATCC No. PTA-5002.

4. A cloning vector which comprises the isolated DNA of claims 1 or 3.

5. A host cell transformed by the cloning vector of claims 2 or 4.

6. A method of producing recombinant OkrAI restriction endonuclease comprising culturing a host cell transformed with the vector of claims 2 or 4 under conditions suitable for expression of said endonuclease.
